(19) United States
(12) Patent Application Publication
(10) Pub. No.: US 2025/0318776 A1
(43) Pub. Date: Oct. 16, 2025

(54) SLEEPING POSITION IDENTIFICATION METHOD, SLEEPING POSITION IDENTIFICATION DEVICE, AND COMPUTER-READABLE STORAGE MEDIUM

(71) Applicant: GOMORE INC., New Taipei City (CN)

(72) Inventor: CHIEN-HAN TSOU, New Taipei City (TW)

(21) Appl. No.: 19/095,634

(22) Filed: Mar. 31, 2025

(30) Foreign Application Priority Data
Apr. 11, 2024 (CN) 202410433809.7

(51) Int. Cl. A61B 5/00

(52) U.S. Cl. CPC A61B 5/4561; A61B 5/4809; A61B 5/6803; A61B 2562/0219

(57) ABSTRACT

An embodiment of the present invention discloses a sleeping position identification method, a sleeping position identification device, and a computer readable storage medium. The method comprises: obtaining a first acceleration signal from a wearable device worn by a user; converting the first acceleration signal into a second acceleration signal based on a transformation function; and determining a current sleeping position of the user based on the second acceleration signal.

### CROSS-REFERENCE TO RELATED APPLICATION

This application claims the priority benefit of China application serial no. 202410433809.7, filed on Apr. 11, 2024. The entirety of China application serial no. 202410433809.7 is hereby incorporated by reference herein and made a part of this specification.

### BACKGROUND

### Technical Field

The present invention relates to a position identification mechanism, and in particular to a sleeping position identification method, a sleeping position identification device, and a computer readable storage medium.

### Description of Related Art

In the prior art, wearable devices can use sensors such as accelerometers, gyroscopes, magnetometers, and techniques such as machine learning and deep learning to identify a user's position. However, the prior art does not disclose a technical solution for determining a sleeping position using a wearable device.

### SUMMARY

In view of the above, the present invention provides a sleeping position identification method, a sleeping position identification device, and a computer readable storage medium, which can be used to solve the above technical problems.

An embodiment of the present invention discloses a sleeping position identification method, applied to a sleeping position identification device, characterized by comprises the following steps. Obtaining a first acceleration signal from a wearable device worn by a user. Converting the first acceleration signal into a second acceleration signal based on a transformation function. Determining the current sleeping position of the user based on the second acceleration signal.

An embodiment of the present invention discloses a sleeping position identification device, characterized by comprising a storage circuit and a processor. The storage circuit stores program code. The processor is coupled to the storage circuit and accesses the program code to execute the following steps. Obtaining a first acceleration signal from a wearable device worn by a user. converting the first acceleration signal into a second acceleration signal based on a transformation function. Determining the current sleeping position of the user based on the second acceleration signal.

An embodiment of the present invention discloses a computer readable storage medium, characterized in that the computer readable storage medium records executable computer programs, the executable computer programs being loaded by the sleeping position identification device to execute the following steps. Obtaining a first acceleration signal from a wearable device worn by a user. Converting the first acceleration signal into a second acceleration signal based on a transformation function. Determining the current sleeping position of the user based on the second acceleration signal.

### BRIEF DESCRIPTION OF THE DRAWINGS

Drawings are included for a further understanding of the present invention, and the drawings are incorporated into this specification and form a part thereof. The drawings illustrate embodiments of the present invention and, together with the description, are used to explain the principle of the invention.

FIG. 1 is a schematic diagram of a sleeping position identification device according to an embodiment of the present invention.

FIG. 2 is a flowchart of a sleeping position identification method according to an embodiment of the present invention.

FIG. 3 is a schematic diagram of an accelerometer according to an embodiment of the present invention.

FIG. 4 is a schematic diagram of a transformation function according to an embodiment of the present invention.

### DESCRIPTION OF THE EMBODIMENTS

Reference will now be made in detail to the exemplary embodiments of the disclosure, examples of which are illustrated in the drawings. Wherever possible, the same reference symbols in the drawings and the description are used to denote the same or similar parts.

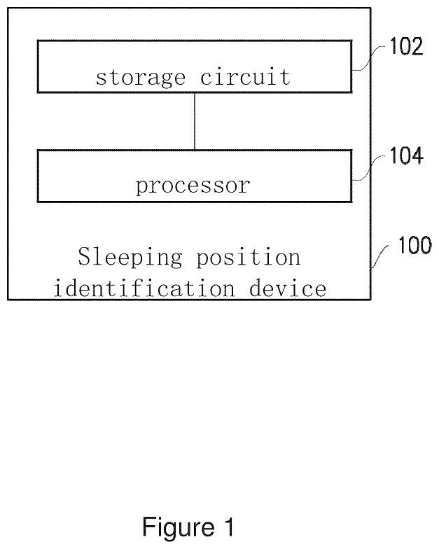

Reference is made to FIG. 1, which is a schematic diagram of a sleeping position identification device according to an embodiment of the present invention. In different embodiments, the sleeping position identification device 100 may be implemented as various smart devices and/or computer devices, but is not limited thereto. In some embodiments, the sleeping position identification device 100 may also be implemented as a wearable device (for example, various earphones) worn by the user, but is not limited thereto.

In some embodiments, the sleeping position identification device 100 may receive an acceleration signal (e.g., a three-axis acceleration signal) measured by the wearable device (e.g., earphones) worn by the user. Furthermore, in embodiments where the sleeping position identification device 100 itself is a wearable device worn by the user, the device 100 may measure the corresponding acceleration signal (e.g., a three-axis acceleration signal) using its built-in accelerometer, but is not limited thereto.

In FIG. 1, the sleeping position identification device 100 includes a storage circuit 102 and a processor 104. The storage circuit 102 is, for example, any type of fixed or portable random access memory (RAM), read-only memory (ROM), flash memory, hard disk, or other similar device or a combination thereof, which can be used to store a plurality of program codes or modules.

The processor 104 is coupled to the storage circuit 102 and may be a general-purpose processor, a special-purpose processor, a conventional processor, a digital signal processor, multiple microprocessors, one or more microprocessors with integrated digital signal processor cores, a controller, a microcontroller, an application-specific integrated circuit (ASIC), a field programmable gate array (FPGA), any other type of integrated circuit, a state machine, an Advanced RISC Machine (ARM)-based processor, or the like.

In the embodiments of the present invention, the processor 104 may access the modules and program code stored in the storage circuit 102 to implement the sleeping position identification method proposed by the present invention; the details are described below.

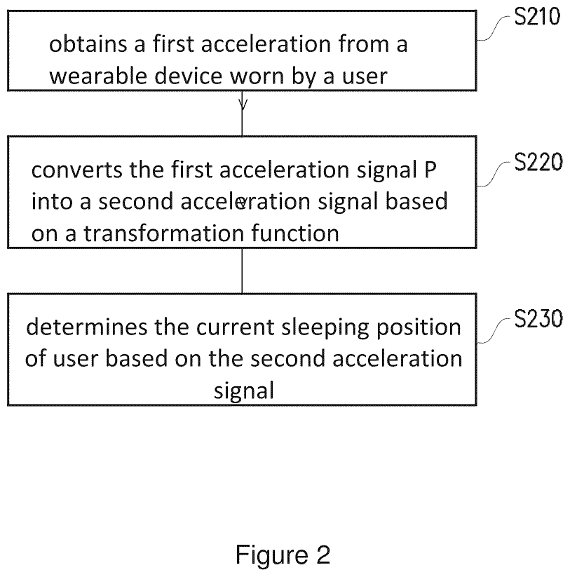

Reference is made to FIG. 2, which is a flowchart of the sleeping position identification method according to an embodiment of the present invention. The method of this embodiment may be executed by the sleeping position identification device 100 of FIG. 1; the details of each step illustrated in FIG. 2 will now be described with reference to the components shown in FIG. 1.

First, in step S210, the processor 104 obtains a first acceleration signal (hereafter denoted by P) from a wearable device (hereafter denoted by B) worn by a user (hereafter denoted by A). For ease of explanation, it is assumed that the sleeping position identification device 100 is the wearable device B worn by user A (for example, earphones). In this case, the processor 104 may, for example, obtain the acceleration signal (e.g., a three-axis acceleration signal) from the built-in accelerometer of the sleeping position identification device 100 as the first acceleration signal P considered in step S210, but is not limited thereto.

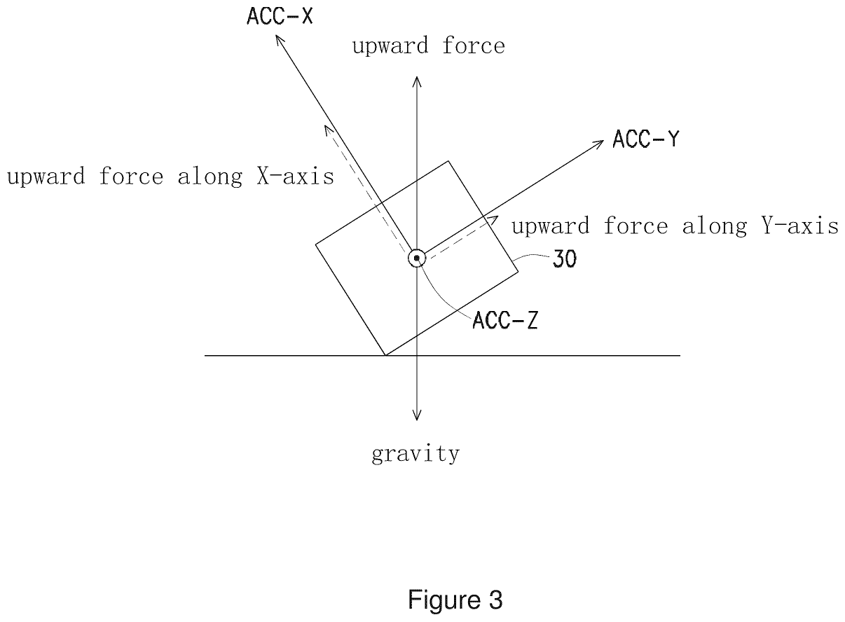

Reference is made to FIG. 3, which is a schematic diagram of an accelerometer according to an embodiment of the present invention. In FIG. 3, the accelerometer 30 is, for example, the built-in accelerometer of the sleeping position identification device 100, and its corresponding coordinate system may be represented by an X-axis (denoted as ACC-X) and a Y-axis (denoted as ACC-Y) as shown in FIG. 3, wherein the Z-axis (denoted as ACC-Z) is, for example, in the direction out of the page, but is not limited thereto. In the embodiments of the present invention, the accelerometer 30 may measure and obtain the upward force counteracting gravity, as well as the components of this upward force along the X-axis, Y-axis, and Z-axis of the accelerometer coordinate system, but is not limited thereto.

In step S220, the processor 104 converts the first acceleration signal P into a second acceleration signal based on a transformation function (hereafter denoted by R).

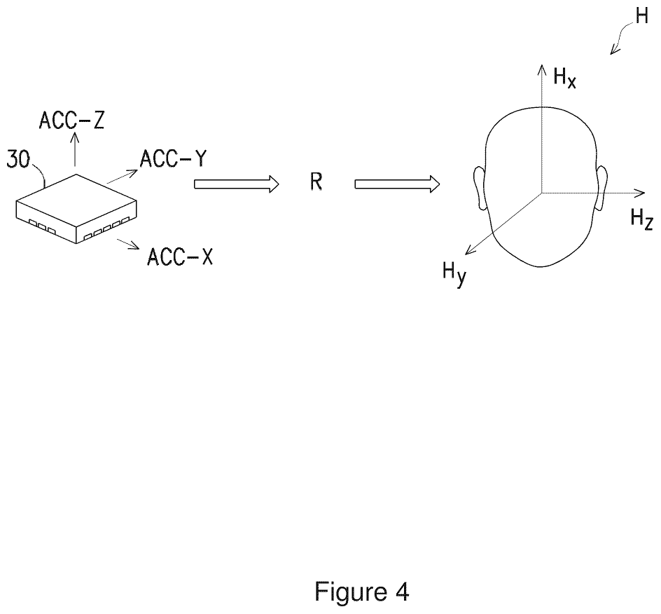

Reference is made to FIG. 4, which is a schematic diagram of the transformation function according to an embodiment of the present invention. In the embodiments of the present invention, in order to correctly identify the sleeping position of user A, it is necessary to first determine the head coordinate system of user A (denoted by H), and based on that, determine the direction of the upward force acting on user A's head in the head coordinate system H. In FIG. 4, the X-axis, Y-axis, and Z-axis of the head coordinate system H are represented as Hₓ, H_{y}, H_{z} respectively. However, since the coordinate system used by the accelerometer 30 of the wearable device (e.g., earphones) is different from the head coordinate system H of user A, it is necessary first to determine, by certain means, a transformation function R that can convert the accelerometer coordinate system into the head coordinate system H of user A, so as to correctly determine the sleeping position of user A.

In different embodiments, the processor 104 may determine the above transformation function R by different methods.

In a first embodiment, the processor 104 may obtain a first reference acceleration signal from a reference wearable device worn by user A, wherein the first reference acceleration signal is detected by the reference wearable device during a first period in which user A maintains a first preset position. In the embodiments of the present invention, the first preset position is, for example, a standing position, but is not limited thereto.
In addition, the processor 104 may obtain a second reference acceleration signal from the reference wearable device worn by user A, wherein the second reference acceleration signal is detected by the reference wearable device during a second period in which user A maintains a second preset position. In the embodiments of the present invention, the second preset position is, for example, a supine position, but is not limited thereto. In different embodiments, the first period and/or the second period may be set to any duration (for example, 30 seconds) according to the designer's requirements, but is not limited thereto.

In a second embodiment, the processor 104 may obtain the first reference acceleration signal from a reference wearable device worn by one or more reference users, wherein the first reference acceleration signal is that detected by the reference wearable device during a first period in which the corresponding reference user maintains a first preset position.

In one embodiment, if there are multiple reference users, the processor 104 may, after obtaining the corresponding first reference acceleration signals from each reference wearable device, take the average value or another statistically computed representative value as the first reference acceleration signal to be considered thereafter, but is not limited thereto.

Similarly, the processor 104 may obtain a second reference acceleration signal from the reference wearable device worn by the one or more reference users, wherein the second reference acceleration signal is that detected by the reference wearable device during a second period in which the corresponding reference user maintains a second preset position.

In one embodiment, if there are multiple reference users, the processor 104 may, after obtaining the corresponding second reference acceleration signals from each reference wearable device, take the average value or another statistically computed representative value as the second reference acceleration signal to be considered thereafter, but is not limited thereto.

In a third embodiment, the processor 104 may also obtain the relevant first and second reference acceleration signals simultaneously by the methods described in the first and second embodiments, but is not limited thereto.

After obtaining the required first and second reference acceleration signals, the processor 104 may, for example, determine the transformation function R based on the first reference acceleration signal and the second reference acceleration signal.

In one embodiment the transformation function R may be represented as:

$$R = {[ \begin{matrix}{V}_{1} \\ {V}_{2} \\ {V}_{3}\end{matrix} ]}^{T} = {[ \begin{matrix}{V}_{stand} \\ {V}_{3} \times {V}_{stand} \\ {V}_{stand} \times {V}_{back}\end{matrix} ]}^{T} \qquad ( 1 )$$

whereV_{stand}is the first unit vector corresponding to the first reference acceleration signal, V_{back}is the second unit vector corresponding to the second reference acceleration signal, and ×denotes the cross product operator.

In one embodiment, the processor 104 may, for example, normalize the first and second reference acceleration signals respectively to determine V_{stand} and V_{back}, and calculate the transformation function R (which, for example, is a transformation matrix) by using equation (1), but is not limited thereto.

After determining the transformation function R, in one embodiment the processor 104 converts the first acceleration signal P (obtained in step S210) into the corresponding second acceleration signal.

In one embodiment, the processor 104 may first convert the first acceleration signal P into a third acceleration signal using the transformation function R (for example, by rotation), and then normalize the third acceleration signal to obtain the second acceleration signal.

In one embodiment, where the first acceleration signal P and the transformation function R are respectively assumed to be a three-axis acceleration signal and a transformation matrix, the third acceleration signal may be represented as “P×R” (for example, a vector). In this case, the second acceleration signal may be represented as:

$$[ \begin{matrix}{a}_{{H}_{x}} & {a}_{{H}_{y}} & {a}_{{H}_{z}}\end{matrix} ] = \frac{P \times R}{‖ P \times R ‖} \qquad ( 2 )$$

where a_{H}_{ₓ}, a_{H}_{_{y}}, a_{Hdz} represent the unit components of the second acceleration signal in the directions of Hₓ, H_{y}, H_{z}, and ∥P×R∥ represents the magnitude of P×R.

After obtaining the second acceleration signal, in step S230 the processor 104 determines the current sleeping position of user A based on the second acceleration signal.

In a fourth embodiment, in response to a determination that a_{H}_{ₓ} is greater than

$$\sqrt{{{a}_{{H}_{y}}}^{2} + {{a}_{{H}_{z}}}^{2}} ,$$

the processor 104 determines that the current sleeping position of user A is a sitting position.

In a fifth embodiment, in response to a determination that a_{H}_{ₓ} is greater than or equal to cos 45°, the processor 104 determines that the current sleeping position of user A is a sitting-sleep position.

In a sixth embodiment, in response to a determination that a_{H}_{ₓ} is not less than cos 45°, a_{H}_{_{z}} equals 0, and a_{H}_{_{y}} is greater than 0, the processor 104 determines that the current sleeping position of user A is a supine position; on the other hand, in response to a determination that a_{H}_{ₓ} is not less than cos 45°, a_{H}_{_{z}} equals 0, and a_{H}_{_{y}} is not greater than 0, the processor 104 determines that the current sleeping position of user A is a prone position.

In a seventh embodiment, in response to a determination that a_{H}_{ₓ} is not less than cos 45°, a_{H}_{_{z}} is not equal to 0, and

$$\frac{{a}_{{H}_{y}}}{\vert {a}_{{H}_{z}} \vert}$$

is greater than or equal to 1, me processor 104 determines that the current sleeping position of user A is a supine position; on the other hand, in response to a determination that a_{H}_{ₓ} is not less than cos 45°, a_{H}_{_{z}} is not equal to 0, and

$$\frac{{a}_{{H}_{y}}}{\vert {a}_{{H}_{z}} \vert}$$

is less than or equal to −1, the processor 104 determines that the current sleeping position of user A is a prone position.

In an eighth embodiment, in response to a determination that a_{H}_{ₓ} is not less than cos 45°, a_{H}_{_{z}} is not equal to 0,

$$\frac{{a}_{{H}_{y}}}{\vert {a}_{{H}_{z}} \vert}$$

has an absolute value less than 1, and a_{H}_{_{z}} is greater than 0, the processor 104 determines that the current sleeping position of user A is a right lateral recumbent position; on the other hand, in response to a determination that a_{H}_{ₓ} is not less than cos 45, a_{H}_{_{z}} is not equal to 0,

$$\frac{{a}_{{H}_{y}}}{\vert {a}_{{H}_{z}} \vert}$$

has an absolute value less than 1, and a_{H}_{_{z}} is less than or equal to 0, the processor 104 determines that the current sleeping position of user A is a left lateral recumbent position.

The present invention also provides a computer-readable storage medium for executing the sleeping position identification method. The computer-readable storage medium is constituted by a plurality of program instructions embedded therein (for example, setup instructions and deployment instructions). These program instructions may be loaded into the sleeping position identification device 100 and executed by the device to perform the aforementioned sleeping position identification method and functions of the sleeping position identification device 100.

In one embodiment, the sleeping position identification method proposed by the present invention may be executed after the sleeping position identification device 100 determines that the user has fallen asleep. The sleeping position identification device 100 may, based on the first acceleration signal, determine whether the user is stable, and thereby determine whether the user has entered a sleep state.

In one embodiment, if the sleeping position identification device 100 determines that the variation amount of the second acceleration signal within a unit time, for example 1 second, exceeds a threshold, then sleeping position identification is not performed, that is, no identification result of the user's sleeping position for that unit time is output.

In one embodiment, the sleeping position identification device 100 may output a sleep report after determining that the user's sleep has ended. The sleep report may include the proportions of various sleeping positions as determined during the sleep state, for example, the percentage of time a single sleeping position occupies relative to the total sleeping position time.

In summary, the embodiments of the present invention disclose a technical solution for identifying the current sleeping position of a user based on an acceleration signal provided by a wearable device on the user. In this manner, the user can gain further understanding of their own sleeping position, thereby enabling improved sleep management.

Finally, it should be noted that the above embodiments are provided solely to illustrate the technical solution of the present invention and are not intended to limit it; although the present invention has been described in detail with reference to the above embodiments, those skilled in the art will understand that modifications or equivalent substitutions may be made to the technical solution recited in the above embodiments, either in part or in whole, without departing from the scope of the present invention.

What is claimed is:

1. A sleeping position identification method, applied to a sleeping position identification device, comprising:
obtaining a first acceleration signal from a wearable device worn by a user;
converting the first acceleration signal into a second acceleration signal based on a transformation function; and
determining a current sleeping position of the user based on the second acceleration signal.

2. The method according to claim 1, further comprising:
obtaining a first reference acceleration signal from at least one reference wearable device worn by the user or at least one reference user, wherein the first reference acceleration signal is that detected by the at least one reference wearable device during a first period in which the user or the at least one reference user maintains a first preset position;
obtaining a second reference acceleration signal from the wearable device worn by the user or the at least one reference user, wherein the second reference acceleration signal is that detected by the at least one reference wearable device during a second period in which the user or the at least one reference user maintains a second preset position; and
determining the transformation function based on the first reference acceleration signal and the second reference acceleration signal.

3. The method according to claim 2, wherein the transformation function is represented by R, wherein:
$$R = {[ \begin{matrix}{V}_{1} \\ {V}_{2} \\ {V}_{3}\end{matrix} ]}^{T} = {[ \begin{matrix}{V}_{stand} \\ {V}_{3} \times {V}_{stand} \\ {V}_{stand} \times {V}_{back}\end{matrix} ]}^{T} ,$$
where V_{stand} is a first unit vector corresponding to the first reference acceleration signal, V_{back}is a second unit vector corresponding to the second reference acceleration signal, and × denotes a cross product operator.

4. The method according to claim 2, wherein the first preset position is a standing position and the second preset position is a supine position.

5. The method according to claim 1, wherein converting the first acceleration signal into the second acceleration signal based on the transformation function comprises:
converting the first acceleration signal into a third acceleration signal using the transformation function; and
normalizing the third acceleration signal to obtain the second acceleration signal.

6. The method according to claim 1, wherein the transformation function is represented by R, the first acceleration signal is represented by P, and the second acceleration signal is represented by [a_{H}_{ₓ} a_{H}_{_{y}} a_{H}_{_{z}}], wherein:
$$[ \begin{matrix}{a}_{{H}_{x}} & {a}_{{H}_{y}} & {a}_{{H}_{z}}\end{matrix} ] = \frac{P \times R}{‖ P \times R ‖}$$

7. The method according to claim 1, wherein the second acceleration signal is represented by [a_{H}_{ₓ} a_{H}_{_{y}} a_{H}_{_{z}}], and determining the current sleeping position of the user based on the second acceleration signal comprises:
in response to a determination that a_{H}_{ₓ} is greater than
$$\sqrt{{{a}_{{H}_{y}}}^{2} + {{a}_{{H}_{z}}}^{2}} ,$$
determining that the current sleeping position of the user is a sitting position.

8. The method according to claim 1, wherein the second acceleration signal is represented by [a_{H}_{ₓ} a_{H}_{_{y}} a_{H}_{_{z}}] and determining the current sleeping position of the user based on the second acceleration signal comprises:
in response to a determination that a_{H}_{ₓ} is greater than or equal to cos 45°, determining that the current sleeping position of the user is a sitting-sleeping position.

9. The method according to claim 1, wherein the second acceleration signal is represented by [a_{H}_{ₓ} a_{H}_{_{y}} a_{H}_{_{z}}] and determining the current sleeping position of the user based on the second acceleration signal comprises:
in response to a determination that a_{H}_{ₓ} is not less than cos 45°, a_{H}_{_{z}} equals 0, and a_{H}_{_{y}} is greater than 0, determining that the current sleeping position of the user is a supine position;
in response to a determination that a_{H}_{ₓ} is not less than cos 45°, a_{H}_{_{z}} equals 0, and a_{H}_{_{y}} is not greater than 0, determining that the current sleeping position of the user is a prone position.

10. The method according to claim 1, wherein the second acceleration signal is represented by [a_{H}_{ₓ} a_{H}_{_{y}} a_{H}_{_{z}}] and determining the current sleeping position of the user based on the second acceleration signal comprises:
in response to a determination that a_{H}_{ₓ} is not less than cos 45°, a_{H}_{_{z}} is not equal to 0, and
$$\frac{{a}_{{H}_{y}}}{\vert {a}_{{H}_{z}} \vert}$$
is greater than or equal to 1, determining that the current sleeping position of the user is a supine position;
in response to a determination that a_{H}_{ₓ} is not less than cos 45°, a_{H}_{_{z}} is not equal to 0, and
$$\frac{{a}_{{H}_{y}}}{\vert {a}_{{H}_{z}} \vert}$$
is less than or equal to −1, determining that the current sleeping position of the user is a prone position.

11. The method according to claim 10, wherein determining the current sleeping position of the user based on the second acceleration signal further comprises:
in response to a determination that a_{H}_{ₓ} is not less than cos 45°, a_{H}_{_{z}} is not equal to 0,
$$\frac{{a}_{{H}_{y}}}{\vert {a}_{{H}_{z}} \vert}$$
has an absolute value less than 1, and a_{H}_{_{z}} is greater than 0, determining that the current sleeping position of the user is a right lateral recumbent position;
in response to a determination that a_{H}_{ₓ} is not less than cos 45°, a_{H}_{_{z}} is not equal to 0,
$$\frac{{a}_{{H}_{y}}}{\vert {a}_{{H}_{z}} \vert}$$
has an absolute value less than 1, and a_{H}_{_{z}} is less than or equal to 0, determining that the current sleeping position of the user is a left lateral recumbent position.

12. The method according to claim 1, wherein the sleeping position identification device is the wearable device.

13. The method according to claim 12, wherein the wearable device is earphones.

14. The method according to claim 1, further comprising:
determining, based on the first acceleration signal, whether the user has entered a sleep state.

15. The method according to claim 1, further comprising:
determining whether a variation amount of the second acceleration signal within a unit time exceeds a threshold; and
in response to a determination that the variation amount exceeds the threshold, not determining the current sleeping position of the user.

16. The method according to claim 1, further comprising:
outputting a sleep report, wherein the sleep report comprises a percentage of time that each individual sleeping position, as determined during a sleep state, occupies relative to a total sleeping position time.

17. A sleeping position identification device, comprising:
a storage circuit for storing program code; and
a processor coupled to the storage circuit and configured to access the program code to execute:
obtaining a first acceleration signal from a wearable device worn by a user;
converting the first acceleration signal into a second acceleration signal based on a transformation function; and
determining a current sleeping position of the user based on the second acceleration signal.

18. A non-transitory computer-readable storage medium, wherein the non-transitory computer-readable storage medium records executable computer programs, and the executable computer programs are loaded by a sleeping position identification device to execute the following steps:
obtaining a first acceleration signal from a wearable device worn by a user;
converting the first acceleration signal into a second acceleration signal based on a transformation function; and
determining a current sleeping position of the user based on the second acceleration signal.
